(19) United States
(12) Patent Application Publication
(10) Pub. No.: US 2006/0231099 A1
(43) Pub. Date: Oct. 19, 2006

(54) SYSTEM AND METHOD FOR MAKING DRY AND HYDROCARBON-FREE COMPRESSED AIR

(75) Inventor: Thomas J. Jeffers, Spring, TX (US)

Correspondence Address: WILLIAM H. EILBERG, THREE BALA PLAZA, SUITE 501 WEST, BALA CYNWYD, PA, 19004, US

(73) Assignee: Generon IGS, Inc., Houston, TX (US)

(21) Appl. No.: 11/277,841

(22) Filed: Mar. 29, 2006

Related U.S. Application Data
(60) Provisional application No. 60/672,886, filed on Apr. 19, 2005.

(51) Int. Cl. A61M 16/00

(52) U.S. Cl. 128/204.25

(57) ABSTRACT

A system for making dry, hydrocarbon-free compressed air includes a compressor, a membrane drier, and a carbon bed connected in series. A moisture separator may also be included to remove liquid water from the stream. A filter may be placed after the carbon bed to remove residual carbon dust. The output of the system can be connected to operate virtually any pneumatic device, such as a control valve or pneumatically-operated instrument. Because the chemical content of the compressed air is not changed, it remains breathable, and does not create a safety hazard.

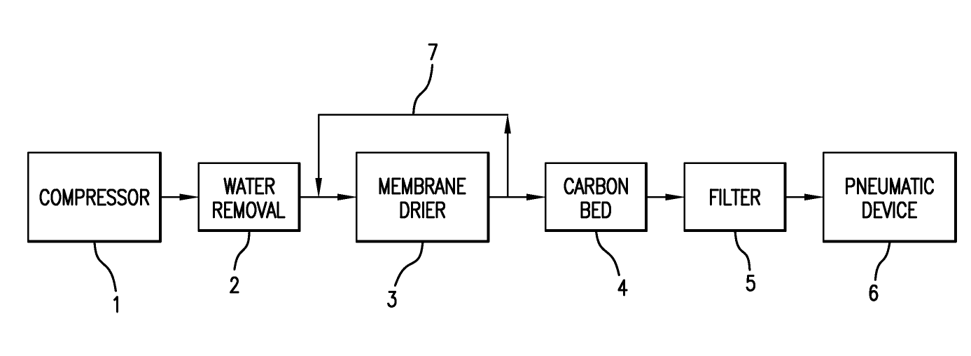

### CROSS-REFERENCE TO PRIOR APPLICATION

Priority is claimed from U.S. provisional patent application Ser. No. 60/672,886, filed Apr. 19, 2005, the disclosure of which is incorporated by reference herein.

### BACKGROUND OF THE INVENTION

This invention relates to the field of treatment of air, and provides a system and method for making compressed air that is substantially free of water and hydrocarbons. The dry and hydrocarbon-free air made by this invention can be used advantageously for driving pneumatically-operated instruments or control valves, or the like, or for providing breathable air in industrial environments.

It has been known to use compressed air to drive devices such as pneumatic valves, or other pneumatically-operated equipment. Compressed air is also used in metal shops, and similar environments, to blow debris away from parts that have been fabricated. It is also used in other industrial applications.

In the above and in many other applications, it is desirable to use compressed air that is both dry and substantially free of hydrocarbons. Residual water, or water vapor, in the compressed air is likely to clog an instrument or control valve or other pneumatically-operated device, and is also likely to cause corrosion. Residual hydrocarbons in the air stream tend to form deposits, especially in crevices in the pneumatic devices, and these deposits degrade the performance of the devices.

A pneumatically-operated device can work with virtually any gas. However, considerations of safety make it preferable to use a breathable gas to operate such a device. If a pneumatic device, such as a control valve, develops a leak, and if the gas operating the device is nitrogen, for example, the leakage of the nitrogen would eventually deplete the proportion of oxygen in the vicinity of the device, causing unknowing workers to be subjected to a hazard. But if one operates the pneumatic device with a breathable gas, any leakage will not pose a danger.

The present invention provides an economical system and method for making a breathable gas that is suitable for operating pneumatic equipment, and which is not likely to cause corrosion or otherwise shorten the useful life of such equipment.

### SUMMARY OF THE INVENTION

The present invention includes a compressor which receives ambient air and compresses it. The compressed air passes through a moisture separator which removes liquid water that may have formed as a result of the compression. The stream then passes through a membrane drier, the principal component of which is a polymeric membrane that exhibits selectivity for water vapor. The dried stream then passes through a carbon bed, which adsorbs hydrocarbons from the stream. A final filter can be used to remove residual carbon dust from the stream. The product gas can then be used to drive a pneumatic device, such as a control valve, or any component which operates with compressed air. Because the air has not been separated into components, it remains breathable, so it can safely be used in a factory or shop.

The present invention therefore has a principal object of providing a system and method for making compressed air that is substantially free of both water vapor and hydrocarbons.

The invention has the further object of providing an economical method for driving a pneumatic component or device.

The invention has the further object of prolonging the useful life of a pneumatic component, through the use of dry, hydrocarbon-free compressed air.

The invention has the further object of providing a system for making dry, hydrocarbon-free compressed air, wherein the system has no moving parts except a compressor.

The invention has the further object of reducing the cost of making dry, hydrocarbon-free compressed air.

The invention has the further object of economically providing a breathable gas that is both dry and substantially hydrocarbon-free.

The reader skilled in the art will recognize other objects and advantages of the present invention, from a reading of the following brief description of the drawing, the detailed description of the invention, and the appended claims.

### BRIEF DESCRIPTION OF THE DRAWING

The FIGURE provides a block diagram of the system of the present invention.

### DETAILED DESCRIPTION OF THE INVENTION

The basic components of the system of the present invention are shown in the FIGURE. Compressor 1 receives ambient air, and compresses it. The compressed air passes through moisture separator 2, which removes liquid water that may have formed when the air is compressed. The moisture separator may also remove liquid oil that may have leaked from the compressor, and which may have entered the process stream. The air then passes through membrane drier 3, where substantially all of the water vapor in the stream is removed.

The dried air passes through carbon bed 4, which adsorbs hydrocarbon particulates in the stream. Such particulates may comprise the residue of oil that has leaked from the compressor into the process stream. The membrane drier is operated with a sweep stream of product gas which is recycled from the exit stream of the drier, as indicated by conduit 7.

The membrane drier has, as its primary component, a polymeric membrane which is chosen to have high selectivity for water vapor. The membrane used in the present invention may be the air dehydration membrane which is described in U.S. patent application Ser. No. 10/640,363, published as US 2005-0034602 A1, and in U.S. patent application Ser. No. 11/185,049, published as US 2005-0252377 A1, the disclosures of which are incorporated by reference herein. Preferably, the membrane drier should remove enough water vapor to reduce the dewpoint of the air to about −70° F. The carbon bed should preferably remove enough hydrocarbon residue to reduce the concentration to less then 10 parts per billion.

A final particulate filter 5 may be used to remove residual carbon dust from the product gas. The filter is preferred, but not absolutely required.

The system may include various other optional components, such as are described in U.S. provisional patent application Ser. No. 60/565,949, filed Apr. 27, 2004, and in a corresponding nonprovisional patent application Ser. No. 11/101,273, filed Apr. 6, 2005, entitled “Air Separation System Using Dehydration Membrane for Pretreatment of Compressed Air”, by inventors John A. Jensvold and Frederick L. Coan, and published as US 2005-0235826 A1. The disclosures of both of the afore-mentioned applications are incorporated by reference herein. In these disclosures, there are various filters, heaters, and/or moisture separators positioned between a compressor and a dehydration membrane. The system of the present invention may include any or all of these components. Note, however, that the system of the present invention differs from the system shown in the above-cited applications, in that the present system does not include a gas separation module. As explained above, the gas used to operate pneumatic instruments or devices should be breathable air, so it is not necessary or desirable to separate the air into nitrogen and oxygen, for the purposes of this invention.

The dry, hydrocarbon-free compressed air exiting carbon bed 4 (or filter 5, if present), comprises the product gas of the system. The product gas can be used in a variety of applications requiring a gas having a low dewpoint and a low level of hydrocarbons.

As shown in the FIGURE, the product gas is directed to pneumatic device 6. Block 6 is intended to represent any pneumatically-operated device, such as a control valve, or laboratory instrument, or virtually any other device that uses a stream of air. Block 6 could also represent a device as simple as a nozzle used to direct compressed air towards a workpiece, such as to blow away debris from machined parts. Or it could represent virtually any other pneumatically-operated component.

The system of the present invention can be set up very quickly, and is entirely self-contained. The system is compact, and requires minimal power and maintenance. Except for the compressor, the system has no moving parts, and can be operated with very little power, for most applications. Prior art technologies, such as systems using refrigeration driers or desiccant driers are more costly to operate because of their higher maintenance costs. A refrigeration drier also consumes far more power than the system of the present invention.

The dry and hydrocarbon-free compressed air, produced by the present invention, can be used in the following applications:

1. Electronics Fabrication

The compressed air of the present invention can be used to blow debris away from electronic components, and for other purposes.

2. Food Transportation

The compressed air of the present invention can be used, for example, in a pneumatic conveyor system, wherein small edible items (such as nuts or snack foods) are directed by an air stream into packages.

3. Laboratories

The compressed air of the present invention can be used as a carrier gas for laboratory instruments such as gas chromatographs, for example.

4. Portable Breathing Air

The compressed air of the present invention could be used in a portable unit for providing breathing air for industrial workers performing tasks in oxygen-depleted environments.

5. Instrumentation

The compressed air of the present invention could be used to operate a variety of instruments, such as control valves, barometers, and others.

The list given above is intended to provide examples of the use of the present invention, and is not intended to limit the scope of the invention. Many other uses of the present invention, in addition to those listed above, are possible.

The present system can be modified in various ways. As indicated above, the specific configuration of components can be changed, according to the needs of a particular application. Additional heaters, filters, moisture traps, etc. could be inserted between the compressor and the device using the product gas. Such modifications should be considered within the spirit and scope of the following claims.

What is claimed is:

1. A system for making dry, hydrocarbon-free compressed air, comprising:
a) a compressor,
b) a membrane drier, the membrane drier being connected to receive compressed air from the compressor,
c) a carbon bed connected to receive dry, compressed air from the membrane drier,
wherein the carbon bed has an output which comprises a stream of dry, compressed air which is substantially hydrocarbon-free.

2. The system of claim 1, further comprising a moisture separator connected between the compressor and the membrane drier.

3. The system of claim 1, further comprising a filter connected to the output of the carbon bed.

4. The system of claim 1, wherein the membrane drier has an output, and wherein a portion of said output of the membrane drier is connected to an input of the membrane drier.

5. The system of claim 1, wherein the output of the carbon bed comprises breathable air.

6. A system for operating a pneumatic device, comprising:
a) a compressor for compressing ambient air,
b) a moisture separator, connected to the compressor, for removing liquid from compressed air produced by the compressor,
c) a membrane drier, connected to an output of the moisture separator, for removing water vapor from the compressed air,
d) a carbon bed, connected to an output of the membrane drier, for removing hydrocarbons from the compressed air,
e) an output of the carbon bed being connected to a pneumatic device such that compressed, dry, hydrocarbon-free air operates the pneumatic device.

7. The system of claim 6, further comprising a filter for removing residual carbon dust from the compressed air, the filter being connected between the carbon bed and the pneumatic device.

8. A method of making dry, hydrocarbon-free compressed air, comprising:
a) compressing ambient air,
b) passing compressed air made in step (a) through a membrane having selectivity for water vapor, so as to dry the compressed air, and
c) passing dried compressed air made in step (b) through an adsorbent so as to remove hydrocarbons from said compressed air.

9. The method of claim 8, wherein step (a) is followed by the step of removing liquid water from the compressed air before passing the compressed air through the membrane.

10. The method of claim 8, wherein step (c) is followed by the step of filtering the compressed air so as to remove residual carbon dust.

11. The method of claim 8, wherein the compressed air exiting the adsorbent comprises breathable air.

12. A method of operating a pneumatic device, comprising:
a) compressing ambient air,
b) passing compressed air through a membrane drier so as to remove water vapor from the compressed air,
c) passing compressed air, exiting the membrane drier, through an adsorbent bed so as to remove hydrocarbons from the compressed air, and
d) using compressed air produced in step (c) to operate a pneumatic device.

13. The method of claim 12, wherein step (a) is followed by the step of removing liquid water from the compressed air before passing the compressed air through the membrane drier.

14. The method of claim 12, wherein step (c) includes the step of filtering the compressed air so as to remove residual carbon dust.

15. The method of claim 12, wherein the compressed air used in step (d) comprises breathable air.
